(19) United States
(12) Patent Application Publication
(10) Pub. No.: US 2022/0031908 A1
(43) Pub. Date: Feb. 3, 2022

(54) Injectable Therapeutic Biocompatible Co-Polymers and Methods of Making and Using Same

(71) Applicant: MOREHOUSE COLLEGE, Atlanta, GA (US)

(72) Inventor: Juana Mendenhall, Austell, GA (US)

(21) Appl. No.: 17/200,540

(22) Filed: Mar. 12, 2021

Related U.S. Application Data
(63) Continuation of application No. 15/574,519, filed on Nov. 16, 2017, now abandoned.
(60) Provisional application No. 62/162,989, filed on May 18, 2015.

(51) Int. Cl. A61L 27/16; A61L 27/52; C08F 251/00; A61K 9/00; A61L 27/38; A61L 27/54; A61K 47/32; A61L 27/50; A61K 47/36; A61L 27/20; B33Y 80/00; C08F 290/10; B33Y 70/00; B33Y 10/00; A61P 19/02

(52) U.S. Cl. CPC A61L 27/16; A61L 2430/24; C08F 251/00; A61K 9/0024; A61L 27/3817; A61L 27/54; A61K 47/32; A61L 27/50; A61K 47/36; A61L 27/20; B33Y 80/00; C08F 290/10; B33Y 70/00; B33Y 10/00; A61P 19/02; A61L 2300/252; A61L 2300/404; A61L 2300/41; A61L 2300/428; A61L 2400/06; A61L 2430/06; A61L 27/52

(57) ABSTRACT

Biocompatible copolymers and thermo-responsive hydrogels formed from the copolymers are disclosed. The biocompatible copolymers include monomers comprising polysaccharides or derivatives thereof, therapeutic agents or derivatives thereof and thermo-responsive monomers and are cross-linked with an acrylamide-containing crosslinker. The hydrogels are used as implant materials to treat or prevent joint damage or osteoarthritis in a subject.

### CROSS-REFERENCE TO RELATED APPLICATIONS

This application is a continuation of U.S. patent application Ser. No. 15/574,519 filed Nov. 16, 2017, which is a national stage entry of PCT/US16/33114 filed May 18, 2016, which claims the benefit of U.S. provisional application No. 62/162,989 filed May 18, 2015.

### BACKGROUND

Osteoarthritis and damaged joints affect millions of people annually. Current medical devices used to treat these problems do not heal or effectively treat diseased joints and do not stay in place over time.

Articular cartilage is the tissue that covers the articular surfaces of bones where they come together to form synovial joints. Articular cartilage helps joints to move by reducing friction between bones. Articular cartilage can become damaged by normal wear and tear or due to injury.

Repairing damage to articular cartilage presents a daunting challenge in tissue engineering and regenerative medicine. Hypoxic conditions increase cartilage defects by affecting healthy cartilage cell growth and regeneration. Articular cartilage is a highly organized avascular tissue that includes cells called chondrocytes that are embedded throughout an extracellular matrix (ECM) of collagens, proteoglycans and noncollagenous proteins. Healthy chondrocytes deposit ECM proteins such as collagen and glycosoaminoglycans in a normal oxygen environment of 20% or higher. However, if oxygen levels are depleted and hypoxia is induced, chondrocytes do not produce the high levels of ECM proteins which contribute to the high mechanical integrity of articular cartilage. This presents a decrease in chondrocyte viability and proliferation needed for regenerating healthy articular cartilage.

Thermo-sensitive hydrogels have been proposed as tissue scaffolds for cartilage tissue regeneration; however, use of those hydrogels in clinical applications has been limited due to poor mechanical stability and poor therapeutic efficiency. Moreover, in diseased or damaged articular cartilage models, known tissue scaffolds do not provide the therapeutic support needed to facilitate viable chondrocyte proliferation.

### SUMMARY

Provided herein are materials for treating defective or diseased articular cartilage. The materials include synthetic biocompatible copolymers useful, for example, when incorporated into injectable therapeutic hydrogels. The hydrogels are thermo-responsive and useful as implant materials.

The biocompatible copolymers include a first plurality of monomers each including a polysaccharide or derivative thereof, a second plurality of monomers each including a therapeutic agent or derivative thereof, a third plurality of monomers each including a thermo-responsive monomer, and an acrylamide-containing cross-linking agent. Optionally, each of the monomers and the cross-linking agent include a vinyl functional group, and those vinyl functional groups form the backbone of the biocompatible copolymer.

Optionally, the polysaccharide or derivative thereof includes hyaluronic acid or a hyaluronic acid derivative (e.g., methacrylated hyaluronic acid or glycidyl methacrylated hyaluronic acid). Optionally, the thermo-responsive monomer includes n-vinylcaprolactam. Optionally, the therapeutic agent or derivative thereof includes an anti-osteoarthritic agent or derivative thereof. Optionally, the anti-osteoarthritic agent or derivative thereof includes vitamin E, α-tocopherol, curcurmin, carvacrol, catechin, or a derivative thereof. Optionally, the acrylamide-containing cross-linking agent includes bisacrylamide or poly(ethyleneoxide diacrylamide).

Optionally, the biocompatible copolymer further includes a surface adhesion protein. Optionally, the surface adhesion protein is an RGD protein.

Also provided herein are hydrogel implant materials including the biocompatible copolymer described herein. The hydrogel implant materials are useful, for example, for treating defective or diseased articular cartilage. Optionally, the hydrogel implant material is a liquid at temperatures below about 32° C. (e.g., about 23° C.). Optionally, the hydrogel implant material is a gel at temperatures above about 32° C. (e.g., between about 32° C. to about 40° C.). Thus, the hydrogel implant can polymerize at temperatures at or above about 32 degrees, and is a gel at body temperature, for example.

Optionally, the hydrogel implant material can further include an additional therapeutic agent. The additional therapeutic agent can be, for example, an anti-inflammatory agent or an anti-infective agent. Optionally, the hydrogel implant material further includes a plurality of chondrocytes. Optionally, the hydrogel implant material can be in the form of a joint meniscus or fragment thereof.

Methods of treating or preventing joint damage or osteoarthritis in a subject are also provided herein. The methods of treating or preventing joint damage or osteoarthritis in a subject include administering to a joint in the subject an effective amount of any of the hydrogel implant materials described herein. Optionally, the joint is a knee joint. Optionally, the composition is administered by intra-articular injection.

Also provided herein are methods of treating or preventing joint damage or osteoarthritis in a subject including administering a hydrogel implant material in the form of a joint meniscus or fragment thereof and wherein the administering step is surgical implantation. Optionally, the joint meniscus or fragment thereof can be produced using three dimensional printing.

Also provided herein are co-polymerizable compositions including n-vinyl caprolactam, methacrylated hyaluronic acid, a therapeutic agent or derivative thereof containing a vinyl functional group, and an acrylamide containing crosslinking agent. Optionally, the co-polymerizable composition also includes a polymerization initiator. Optionally, the polymerization initiator is a water soluble polymerization initiator (e.g., 2,2′-azobis[N-(2-carboxyethyl)-2-methylpropionamidine]hydrate). Alternatively, the polymerization initiator is a water insoluble polymerization initiator (e.g., azobisisobutyronitrile). Optionally, the therapeutic agent or derivative thereof is an anti-osteoarthritic agent or derivative thereof (e.g., vitamin E, α-tocopherol, curcumin, carvacrol, catechin, or derivatives thereof). Optionally, the acrylamide-containing crosslinking agent is bisacrylamide or poly(ethyleneoxide diacrylamide).

Further provided herein are methods for making a hydrogel implant, comprising: placing any of the co-polymerizable compositions described herein within a three-dimensional printing device and polymerizing the co-polymerizable composition in one or more printing cycles to produce a hydrogel implant.

### BRIEF DESCRIPTION OF THE DRAWINGS

FIGS. 1A-C, panel A (left panel) is a picture of a biocompatible composite gel prepared from 85% n-vinylcaprolactam (PVCL) and 15% methacrylated hyaluronic acid (meHA). FIG. 1, panel B (middle panel) is a picture of a biocompatible composite gel prepared from 15% PVCL and 85% meHA. FIG. 1, panel C (right panel) is a picture of a biocompatible composite gel prepared from 50% PVCL and 50% meHA.

FIGS. 2A-B, panel A (left panel) is a picture of a biocompatible composite gel prepared from methacrylated hyaluronic acid (meHA) and n-vinylcaprolactam (PVCL) being three-dimensionally (3D) printed at room temperature. FIG. 2, panel B (right panel) is a picture of a biocompatible composite gel prepared from meHA and PVCL being 3D printed at a lower critical solution temperature (LCST), depicting the cloudy point (opaque) phase change.

FIGS. 3A-E contains graphs showing turbidity (i.e., cloudy point) measurements of meHA and PVCL biocompatible composite hydrogels at 2% (w/v) concentration. Panel A shows the turbidity measurement of meHA. Panel B shows the turbidity measurement of PVCL. Panel C shows the turbidity measurement of PVCL-g-HAL. Panel D shows the turbidity measurement of PVCL-g-HA2. Panel E shows the turbidity measurement of PVCL-g-HA3.

FIGS. 4A-D contains graphs showing cell viability measurements of C28/12 human chondrocytes cells in hydrogels performed under normal conditions (20% O₂) after 24 hours, 3 days, and 7 days and under hypoxia (5% O₂) after 24 hours.

FIGS. 5A-I, left panels (top to bottom), show the DNA, GAG, and hydroxyproline metabolism profiles for cells cultured in PVCL at 20% and 1% O₂ levels. FIG. 5, middle panels (top to bottom), show the DNA, GAG, and hydroxyproline metabolism profiles for cells cultured in meHA at 20% and 1% O₂ levels. FIG. 5, right panels (top to bottom), show the DNA, GAG, and hydroxyproline metabolism profiles for cells cultured in a PVCL-g-HA-aTA (labeled as JD4) at 20% and 1% O₂ levels.

FIG. 6A-F, left panels (top to bottom), show the DNA (panel a), GAG (panel c), and hydroxyproline (panel e) metabolism profiles for cells cultured in PVCL-g-HA1 at 20% and 1% O₂ levels. FIG. 6, right panels (top to bottom), show the DNA (panel b), GAG (panel d), and hydroxyproline (panel f) metabolism profiles for cells cultured in meHA at 20% and 1% O₂ levels.

FIGS. 7A-D are SEM images of (A) PVCL-g-HA1, (B) enhanced PVCL-g-HA1, (C) PVCL-g-HA3, and (D) PVCL-g-HA3.

FIGS. 8 A-E are graphs showing elastic modulus (G′) versus temperature from temperature ramp rheology experiments for PVCL-g-HA1, PVCL-g-HA2, PVCL-g-HA3, PVCL6, PVCL8, and meHA samples at (A) 1% (w/v), (B) 2% (w/v), (C) 3% (w/v), (D) 4% (w/v), and (E) 5% (w/v).

FIGS. 9 A-E are graphs showing viscous modulus (G″) versus temperature from temperature ramp rheology experiments for PVCL-g-HA1, PVCL-g-IHA2, PVCL-g-HA3, PVCL6, PVCL8 and meHA samples at (A) 1% (w/v), (B) 2% (w/v), (C) 3% (w/v), (D) 4% (w/v), and (E) 5% (w/v).

FIGS. 10 A-C show results of cell viability analyses of average living cells using calcein AM and EthD-1 of (A) PVCL-g-HA1 at 20% and 1%, (B) meHA at 20% and 1%, and (C) percentage of cell viability at normoxia and hypoxia from fluorescent images of green cells/red cells.

FIGS. 11 A-F show DNA and chondrocyte ECM synthesis on PVCL-g-HA and meHA hydrogels at 20% and 1% oxygen levels.

The details of one or more embodiments are set forth in the description below. Other features, objects, and advantages will be apparent from the description and from the claims.

### DETAILED DESCRIPTION

Described herein are materials for treating defective or diseased articular cartilage. The materials include synthetic biocompatible copolymers useful, for example, as injectable, therapeutic hydrogels. The hydrogels are capable of supporting healthy cell growth and thus function as effective biomaterial scaffolds.

### Biocompatible Copolymers and Co-Polymerizable Compositions

The biocompatible copolymers described herein include at least three distinct co-monomers and a cross-linking agent. The biocompatible copolymers include a first co-monomer that includes a polysaccharide or derivative thereof, a second co-monomer that includes a therapeutic agent or derivative thereof, a third co-monomer that is a thermo-responsive monomer, and an acrylamide-containing cross-linking agent.

The first co-monomer includes a polysaccharide or derivative thereof. The first co-monomer is sometimes referred to herein as the polysaccharide monomer. Optionally, the polysaccharide or derivative thereof includes at least one vinyl functional group. Optionally, the polysaccharide or derivative thereof is hyaluronic acid or a hyaluronic acid derivative. For example, the hyaluronic acid derivative can be a methacrylated hyaluronic acid or a glycidyl methylacrylated hyaluronic acid. Optionally the polysaccharide or derivative thereof has a molecular weight (Mw) between about 10 kDa and about 60 kDa (e.g., from about 20 kDa to about 50 kDa or from about 25 kDa to about 45 kDa). The polysaccharide monomer can be present in the biocompatible copolymer in amounts of from about 1 to about 60% weight/volume (e.g., from about 2% to about 45% weight/volume).

The second co-monomer includes a therapeutic agent or derivative thereof. The second co-monomer is sometimes referred to herein as the therapeutic agent monomer. Optionally, the therapeutic agent or derivative thereof includes at least one vinyl functional group. Optionally, the therapeutic agent or derivative thereof is an anti-osteoarthritic agent or derivative thereof. Optionally, the anti-osteoarthritic agent or derivative thereof includes vitamin E, α-tocopherol, curcumin, carvacrol, catechin, a derivative of one of the forgoing, or mixtures of these. As examples, the anti-osteoarthritic agent or derivative thereof may be α-tocopherol acrylate or epigallocatechin gallate. Examples of therapeutic agents that can optionally be used as the second co-monomer include Compounds 1-9 shown below:

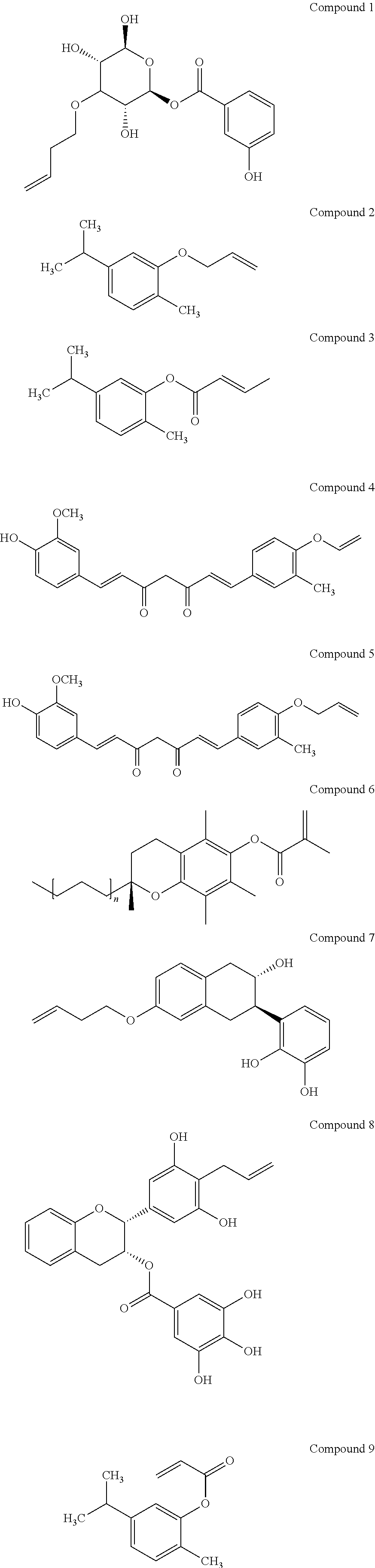

In Compound 6, n is from 1 to 20.

The compounds described above can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Variations on Compounds 1-9 include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Exemplary methods for synthesizing compounds as described herein are provided below in Schemes 1-5. Protective groups (such as Boc) can be used to control the hydroxyl substituent reactivity. Hydroxyl functionalization reactions can be performed to prepare the compounds.

Compound 3 can be prepared according to Scheme 1 shown below:

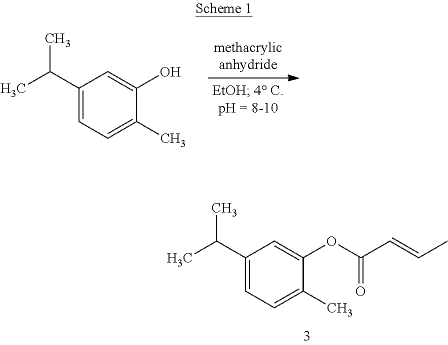 [SMILES: Cc1ccc(C(C)C)cc1O ; C/C=C/C(=O)Oc1cc(C(C)C)ccc1C]

Compound 5 can be prepared according to Scheme 2 shown below:

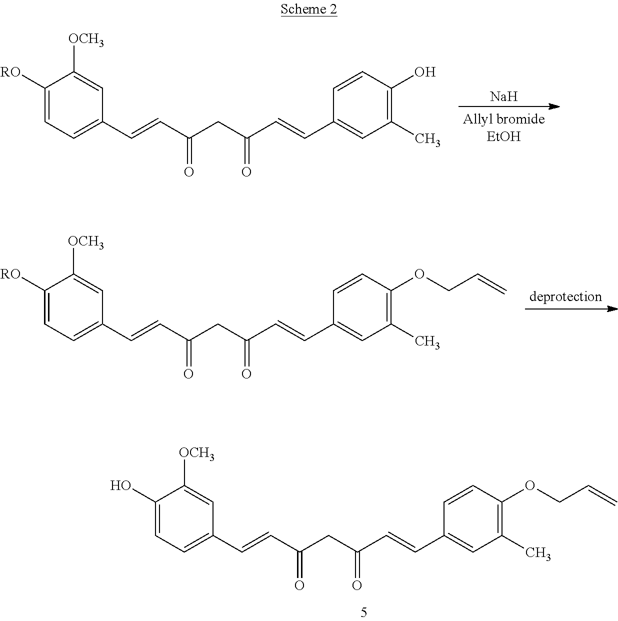

In Scheme 2, R is a protecting group.

Compound 6 can be prepared according to Scheme 3 shown below:

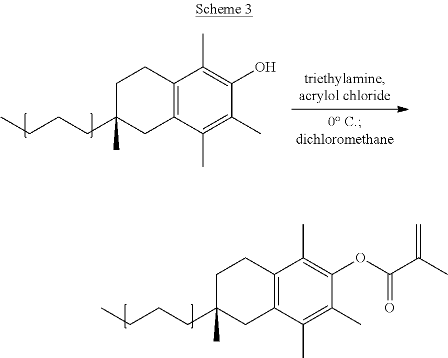

Compound 7 can be prepared according to Scheme 4 shown below:

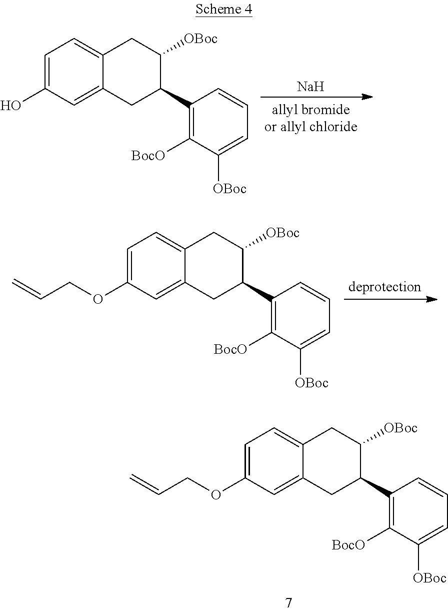

Compound 8 can be prepared according to Scheme 5 shown below:

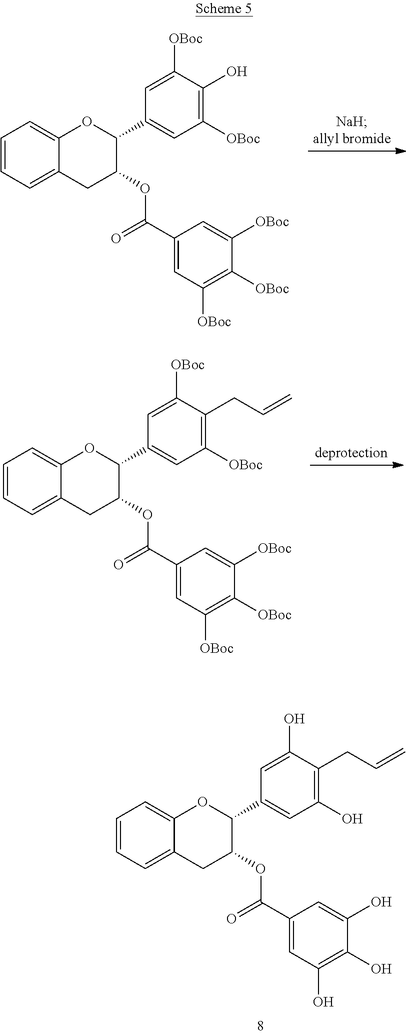

The therapeutic agent monomer can be present in the biocompatible copolymer in amounts of from about 10% to about 40% weight/volume (e.g., from about 10% to about 20% or from about 10% to about 15% weight/volume).

The third co-monomer is a thermo-responsive monomer. Optionally, the thermo-responsive monomer includes at least one vinyl functional group. As used herein, a thermo-responsive monomer is a monomer that when polymerized or co-polymerized forms a thermo-responsive polymer or co-polymer. A thermo-responsive polymer or co-polymer has a lower critical solution temperature (LCST) such that the polymer or copolymer's molecular conformation changes from linear chain/coil to condensed globule upon a temperature change from below the LCST to above the LCST. LCST is a tunable property than can be altered by monomer feed concentration. Optionally, the thermo-responsive monomer is n-vinylcaprolactam. The thermoresponsive monomer is present in the biocompatible copolymer in amounts of from about 10 to about 60% weight/volume (e.g. from about 15 to about 40% weight/volume).

The co-polymer is cross-linked by an acrylamide-containing crosslinking agent. Optionally, the crosslinking agent includes N,N′-methylenebisacrylamide (“bisacrylamide”), poly(ethyleneoxide diacrylamide), poly(ethyleneoxide diacrylate), ethylene glycol dimethacrylate, or mixtures of these. The crosslinking agent car be present in the biocompatible copolymer in amounts of from about 0.1% to about 2.5% weight/volume (e.g. from about 0.5% to about 2.0% or from about 0.5% to about 3.0% weight/volume). Crosslinking density is affected by solvent preference; crosslinking efficiency is affected by selection of radical initiator; and selection of water soluble initiator or organic solvent initiator affects chemical properties (e.g., LCST, swelling ratio) pf the gel.

Optionally, each of the co-monomers and the acrylamide crosslinking agent includes a vinyl functional group, and when polymerized, the vinyl functional groups form the backbone of the biocompatible copolymer. Moreover, the crosslinking agent can include more than one vinyl functional group, and when polymerized with the co-monomers, the vinyl functional groups can be incorporated into the backbones of different copolymers crosslinking the copolymers.

Optionally, the biocompatible copolymer can be functionalized to include a surface adhesion protein. Optionally, the surface adhesion protein may be an RGD protein. For example, the RGD peptide can be attached to the end of the biocompatible polymer using bioconjugation chemistry, such as by using 1-ethyl-3-(3-dimethylaminopropyl)carbodiimide HCl and N-hydroxysuccinimide (EDC/NHS). The procedure for performing the bioconjugation reaction would be understood by a person of ordinary skill in the art. Optionally, the surface adhesion protein can be present in amounts of from about 10% to about 25% weight/volume.

Also provided herein are co-polymerizable compositions including n-vinyl caprolactam, methacrylated hyaluronic acid, a therapeutic agent containing a vinyl functional group, and an acrylamide containing crosslinking agent. Optionally, the co-polymerizable composition also includes a polymerization initiator. Optionally, the polymerization initiator can be is a water soluble polymerization initiator. Exemplary water soluble polymerization initiators include 2,2′-azobis[N-(2-carboxyethyl)-2-methylpropionamidine]hydrate, which is commercially available under the tradename VA-057 from Wako Specialty Chemicals (Osaka, Japan), and 2,2′-azobis(2-methylpropionamidine)dihydrochloride, which is commercially available under the tradename V-50 from Wako Specialty Chemicals. Alternatively, the polymerization initiator is a water insoluble polymerization initiator (e.g., azobisisobutyronitrile (AIBN)). Optionally, the therapeutic agent is an anti-osteoarthritic agent or derivative thereof (e.g., vitamin E, α-tocopherol, curcumin, carvacrol, catechin or derivative thereof). Optionally, the therapeutic agent has been modified to include a vinyl functional group. Optionally, the acrylamide-containing crosslinking agent is bisacrylamide or poly(ethyleneoxide diacrylamide) (PEO-DA).

The co-polymerizable composition can be polymerized in the presence of a solvent. The solvent can be an aqueous solvent or an organic solvent. Optionally, the solvent is water. Optionally, the solvent is benzene.

### Hydrogel Implant Materials

Also provided herein re hydrogel implant materials including the biocompatible copolymer described above. The hydrogel implant materials are useful for treating defective or diseased articular cartilage. The hydrogel implant materials are capable of supporting healthy cell growth and thus function as effective biomaterial scaffolds.

Optionally, the hydrogel implant material includes the biocompatible co-polymer and water. Optionally, the hydrogel implant material is a liquid at temperatures below about 32° C. (e.g., about 23° C.). The liquid can be a viscous liquid. Optionally, the hydrogel implant material is a gel at temperatures above about 32° C. (e.g., between about 32° C. to about 40° C., such as 37° C.). The gel can be characterized as a material that is a hardened, stronger, or mechanically robust form of the liquid material.

Optionally, the hydrogel implant material can further include an additional therapeutic agent. The additional therapeutic agent can be an anti-inflammatory agent (e.g., curcurmin, carvacrol, epigallocatechin gallate, alpha-tocopherol, and (+) cathecin). Optionally, the additional therapeutic agent can be an anti-infective agent (e.g., proflavine).

Optionally, the hydrogel implant material may further include a plurality of chondrocytes, as described below in Example 4. The chondrocytes can be isolated from one or more subject, including for example, a mammalian subject. Alternatively, the chondrocytes can be derived from an immortalized cell line (e.g., cell lines such as C-28/12, T/C-28a2, and T/C-28a4). Optionally, the chondrocytes are harvested from the donor subject prior to incorporation into the implant and are administered back to the same or different recipient subject or subjects. The recipient and donor can be the same of different species.

Optionally, the hydrogel implant material may be in the form of a joint meniscus or fragment thereof. The joint meniscus or fragment thereof may by for any joint that has damaged, diseased, or defective articular cartilage, but optionally is for a knee joint.

Also, provided herein are methods for making a hydrogel implant, including placing any co-polymerizable composition described herein within a three-dimensional printing device and polymerizing the co-polymerizable in one or more printing cycles to produce a hydrogel implant.

### Methods of Use

Also provided herein are methods of treating or preventing joint damage or osteoarthritis in a subject. The methods of treating or preventing joint damage or osteoarthritis in a subject include administering to a joint in the subject an effective amount of any of the hydrogel implant materials described herein. The joint may be any joint in need of treatment or prevention of joint damage or osteoarthritis, but optionally, the joint is a knee joint. Optionally, the composition is administered by intra-artic lar injection.

Also provided herein are methods of treating or preventing joint damage or osteoarthritis in a subject including administering a hydrogel implant material in the form of a joint meniscus or fragment thereof and wherein the administering step is surgical implantation. Optionally, the joint meniscus or fragment thereof can be produced using three dimensional printing. Optionally, the printing speed for 3D printing is in the range of 30-300 mm/s (e.g., 50 mm/s, 100 mm/s, 150 mm/s, 200 mm/s, 250 mm/p). Optionally, the dispensing pressure for the 3D printing is in the range of 1-60 psi (e.g., 10, 20, 30, 40, 50 psi). Optionally, the nozzle diameter for the 3d printing is 12, 30, 100, or 200 lam. Optionally, the unsupported length during 3D printing is in the range of 10 to 500 μm (e.g., 50, 100, 150, 200, 250, 300, 350, 400, 450 μm). The dispensing/substrate temperature is hydrogel dependent, but optimal dispensing/substrate temperature could be determined by a person ordinarily skilled in the art without undue experimentation.

Any of the methods of treating or preventing joint damage or osteoarthritis can include the use of any of the hydrogel implants described herein. For example, the hydrogel implant optionally comprises chrondrocytes (e.g., from a donor or cell line), and the hydrogel implant is administered to the subject (the recipient). Optionally chondrocytes are harvested from the subject that receives the implant (autotransplant) or a different subject of the same species (allotransplant) of a different subject of a different species (xenotranplant). Harvested chondrocytes are optionally cultured prior to incorporation into the hydrogel implant.

As used herein the terms treatment, treat, or treating refer to a method of reducing one or more symptoms of a disease or condition. Thus in the disclosed method, treatment can refer to a 10%, 20%, 30%, 4d %, 50%, 60%, 70%, 80%, 90%, or 100% reduction in the severity of one or more symptoms of the disease or condition. For example, a method for treating a condition is considered to be a treatment if there is a 10% reduction in one or more symptoms or signs of the condition in a subject as compared to a control. As used herein, control refers to the untreated condition. Thus the reduction can be a 10%, 20%, 30%, 40%, 50%, 60%, 70%, 80%, 90%, 100%, or any percent reduction in between 10% and 100/as compared to native or control levels. It is understood that treatment does not necessarily refer to a cure or complete ablation of the disease, condition, or symptoms of the disease or condition.

As used herein, the terms prevent, preventing, and prevention of a disease or condition refer to an action, for example, administration of a composition or therapeutic agent, that occurs before or at about the same time a subject begins to show one or more symptoms of the disease or condition, which inhibits or delays onset or severity of one or more symptoms of the disease or condition.

As used herein, references to decreasing or reducing include a change of 10%, 20%, 30%, 40%, 50%, 60%, 70%, 80%, 90% or greater as compared to a control level. Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs; and goats. Non-mammals include, for example, fish and birds.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### Examples

### Materials

N-vinylcaprolactam, azobisisobutyronitile (AIBN), and ethacrylic anhydride were purchased from Sigma Aldrich (St. Louis, Mo.). N-vinylcaprolactam was recrystallized in benzene or acetone. The water soluble free radical initiator, 2,2′azobis[N-2-carboxyethyl)-2-methylpropionamidine]hydrate, VA-057, were purchased from Wako Chemical USA (Richmond, Va.). Hyaluronic acid (sodium hylauronan) was purchased from Lifecore Biomedical, LLC (Chaska, Minn.).

### Methods

### Nuclear Magnetic Resonance Imaging

¹H NMR spectra were carried out using a Bruker 400 MHz spectrometer. Samples were dissolved in deuterated chloroform (CDCl₃) or deuterated water (D₂O) at room temperature.

### Attenuated Transmission Fourier Transform Infrared Spectroscopy (ATR-FTIR)

ATR-FTIR spectra were obtained using a Perkin Elmer spectrometer (Perkin Elmer Corp., Madison, Wis.). Each measurement had 256 scans with a resolution of ±4 cm⁻¹ using a germanium crystal at an incident angle of 45°. The depth of IR penetration at this incident angle was 0.1-1 μm.

### Turbidity

Turbidity measurements were evaluated using a customized set-up containing a water-jacketed beaker on a stir plate. Laser intensity was recorded using a 632 nm helium neon laser and recorded using a handheld detector. A circulating water bath, which was connected to the water-jacketed beaker, was used to control temperature throughout the experiment. Samples were prepared at 2% (w/v) using Millipore (18Ω) water and placed in the middle of the beaker for analysis. Turbidity measurements were recorded between 20.0° C.-40.0° C., and the LCST and gelation temperature (T_{G}) were determined.

### Scanning Electron Microscopy

Lyophilized hydrogels were imaged using a LEICA 400 SEM using 10 kV (LEICA Microsystems, Buffalo Grove, Ill.). All samples were supper coated with gold/palladium.

### Rheology

A TA Instruments AR-G2 rheometer (TA Instruments, New Castle, Del.) was used in dynamic oscillation mode with a parallel plate setup. Hydrogels were prepared using DI water at 1-5% (w/v). The angular frequency of oscillation was varied from 100-0.01 rad/s at constant temperature of 20° (and constant 3% strain (except for 1% concentration and 1% strain). The % strain delivered by the oscillating top plate was varied from 0.1-10% at a constant temperature of 20° C. and constant angular frequency of 1 rad/s. The temperature was varied from 20° C.-45° C. using constant angular frequency of 1 rad/s and constant 3% strain (except for 1% concentration and 1% strain). Gap size changed with sample concentration ranging from 900 μm to 1900 μm.

### Chondrocyte Isolation and Tissue Culture

Articular cartilage samples were harvested from the joint of a fetal bovine calf leg between 5-10 days of age. The samples were washed in phosphate buffer saline (PBS) with 2% antibiotics for 30-60 minutes. Next, samples were cut into smaller pieces then digested in 0.1% collagenase for 16-18 hours at 37° C. in an incubator. The chondrocytes were washed afterwards with PBS. All hydrogel samples (n=4) were sterilized using UV radiation for 16-18 hours. All samples were then mixed in low glucose DMEM supplemented with 10% FBS, 25 μg/mL _{L}-ascorbic acid, and antibiotics at 10% (w/v) 3.65 million cells/milliliter. Low oxygen conditions were employed at 1% oxygen levels using a low oxygen chamber (Heracell 150i, Thermo Fish r Scientific, Waltham, Mass.).

### Cell Viability

Cell viability was checked using fluorescent microscopy imaging of calcein AM and ethidium homodimer-1(EthD-1) kit according to manufactures protocols. First, hydrogel samples (n=4) were washed with PBS at 37° C. then incubated with 2 mM calcein AM and 4 mM EthD-1 for 40 minutes at 37° C. Cell viability was analyzed on Day 1, Day 3, Day 10 and cell viability percent was obtained using Image J analysis. Fluorescent microscopic images were acquired using a Nikon TE 200 Microscope (Nikon Instruments Inc., Melville, N.Y.) equipped with a Spot Junior camera (Micro Video Instruments, Avon, MA).

### Biochemical Assays

Extracellular matrix deposition, collagen, DNA, and sulfated glycosaminoglycans (GAG), were harvested from hydrogel samples (n=4) on day(s) 1, 3, 7, and 10. The dimethylmethylene blue (DMMB) dye assay was employed to quantify GAG content on weight and dry hydrogel samples. DNA content was evaluated using the Hoechst dye assay according to manufacture protocols. Collagen content was determined using the hydroxyproline assay, after acid hydrolysis and reaction with p-dimethylaminobenzaldehyde and chloramine T according to previous methods. Samples were analyzed using the dry and wets for each biochemical analysis.

### Statistical Analysis

Statistical analysis was performed for cell viability, GAG content, DNA, and hydroxyproline was performed using ANOVA: single factor analysis. Statistical significance of these correlations was determined by Tukey post-hoc to determine statistical significance where p<0.05. Samples sets (n=4) were reported as mean±SD for each data set.

### Example 1. Synthesis of meHA/PVCL/Therapeutic Agent Co-Polymer

An injectable hydrogel of meHA/PVCL/α-tocopherol acrylate was prepared using the following method. Vinylcaprolactone (VCL) was recrystallized using acetone and benzene. Methacrylated hyaluronic acid (meHA) was prepared by adding hyaluronan to methacrylic anhydride in about a 20 fold excess and adjusting to pH 8. The solution was allowed to react overnight at 4° C., dialyzed against water for 48 hours, and then lyophilized. The VCL, meHA, and α-tocopherol acrylate (aTA) were placed in deionized water and heated to 55° C. The mixture was allowed to react for 24 hours. The copolymer was precipitated in cold ethanol, dialyzed in deionized water for 48 hours, and lyophilized for 24 hours to yield a white/yellowish precipitate.

### Example 2. Synthesis of meHA/PVCL/Therapeutic Agent Co-Polymer with RGD Protein

Synthesis of Methacrylated Hyaluronic Acid (meHA)

Hyaluronan (MW 60 kDA & 1100 kDA; 2% (w/v)) was added to methacrylic anhydride in a 20 fold excess adjusting to pH 8. The solution was allowed to react overnight at 4° C., dialyzed against water for 48 hours, and then lyophilized. meHA samples were rehydrated at 2% or 5% (w/v) for chemical analysis or characterization.

### Synthesis of Glycidyl Methacrylate-HA (GMHA)

Glycidyl methacrylato-HA (GMHA) was prepared as follows: 0.30 g HA was dissolved in 30 mL distilled water. Triethylamine (1.1 mL), 1.1 mL glycidyl methacrylate (GM), and 1.1 g tetrabutyl ammonium bromide were each added individually to the HA/water solution and allowed to mix completely before adding the next component. The mixture was allowed to react overnight. The mixture was then incubated at 60° C. for an hour. The solution was allowed to cool and was precipitated in acetone. The GMHA product was then rinsed with distilled water to remove any impurities and placed on a lyophilizer to be dehydrated. The product was confirmed using ¹H-NMR spectroscopy (D₂O).

The GMHA hydrogel structure was analyzed by performed a swelling ratio experiment using solvent. Hydrogel swelling ratio, based on mass, was calculated by dividing the total mass after swelling by the dry mass of the gel.

### Synthesis of PVCL-Graft-HA (PVCL-g-HA1, PVCL-g-HA2, and PVCL-g-HA3)

Preparation of PVCL-graft-meHA: VCL was recrystallized in benzene and dried under vacuum. Under an inert environment, recrystallized VCL monomer was allowed to mix with meHA in Millipore (80Ω) water at 55° C. After temperature stabilization, VA-057 (an initiator) was added in dropwise into the dissolved VCL/meHA mixture and allowed to react overnight. The mixture was precipitated in cold ethanol and dialyzed in DI water for up to 7 days followed by freeze-drying.

### Synthesis of PVCL-Graft-GMHA3 (PVCL-g-gmHA3)

The preparation of PVCL-g-gmHA3 was carried out by mixing recrystallized VCL monomer and previously prepared GMHA (1.3×10⁴ Da) in a flask in water under argon gas and heated to 60° C. After 20 minutes, the initiator (VA-057) was added and the reaction was carried out for one hour. The solution was precipitated in an ethanol/water solution and dialyzed against DI water for up to seven (7) days then freeze-dried.

### Synthesis of PVCL-Crosslinked-GMHA (PVCL-c-gmHA5 & PVQL-c-gmHA6)

PVCL-c-GMHA5 and PVCL-c-GMHA6 were prepared by mixing recrystallized VCL monomer with GMHA (9.0×10⁴ Da), adding 0.1 g of bisacrylamide in water (under argon gas), and then heating to 60° C. After 20 minutes, VA-057 (0.05 g) was added and allowed to react between 30 minutes to 3 hours. After the reaction the mixture was precipitated in ethanol/water solution, dialyzed for up to seven (7) days, then freeze-dried.

### Synthesis of PVCL-Crosslinked-HA-Crosslinked-aTA (PVCL-c-HA-c-aTA1 and PVCL-c-HA-c-aTA2)

PVCL-c-HA-c-aTA1 was prepared according to the following procedure: 0.4 g of the meHA sample prepared as described above, 0.398 g aTA, 0.544 g VCL, 0.351 g PEO-DA, and 0.218 g ATBN were dissolved in 30 mL of benzene under argon gas. The reactants were allowed to mix for twenty minutes before being heated to 70° C. for an additional 20 minutes, also under argon gas. Following gelation, the gel was precipitated in cold hexane or ethanol. The mixture was then decanted. The resulting precipitate was dried via vacuum, then dialyzed in DI water for up to seven days and placed on the lyophilizer for 48 hours. The gel was then characterized via ¹H-NMR spectroscopy.

To prepare PVCL-c-HA-c-aTA2 with water as the solvent, 0.478 g meHA was dissolved in 30 mL distilled water followed by aTA (0.4912 g), 0.5744 g VCL, 0.371 g PEO-DA, and 0.2323 g VA-057. The components were mixed. Once dissolved, the solution was heated at 60° C. for 40 minutes. Once the reaction was complete; the gel was precipitated in acetone, allowed to dialyze, and placed on the lyophilizer for 48 hours.

### Graft-Copolymerization of PVCL-Graft-HA-aTA(JD4)

A flask was charged with argon gas, and to the flask was added recrystallized VCL, aTA, and MeHA in deionized water (aTA well mixed). The mixture was heated to 55° C. VA-057 (0.5% (w/v)) in deionized water was added dropwise to the flask. The reaction was carried out for 24 hours, and the product was precipitated in cold ethanol, dialyzed in deionized water for 48 hours, and then lyophilized for 24 hours to yield a yellowish-white fibrous powder.

### Example 3. Exemplary Hydrogels

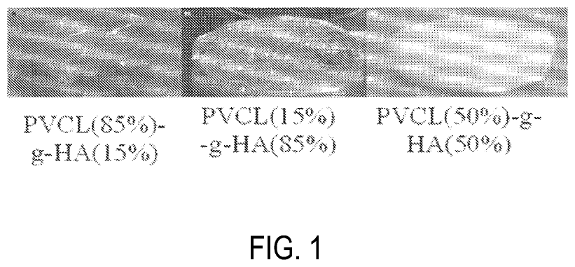

Exemplary hydrogels are shown in FIG. 1. The hydrogel shown in FIG. 1, Panel A is prepared with a graft copolymer including 85% PVCL and 15% HA. The hydrogel shown in FIG. 1, Panel B is prepared with a graft copolymer including 15% PVCL and 85% HA. The hydrogel shown in FIG. 1, Panel C is prepared with a graft copolymer including 50% PVCL and 50% HA. All hydrogels were prepared at room temperature. The pictures in FIG. 1 show the effects on gel consistency that result from changing the percentages of monomers.

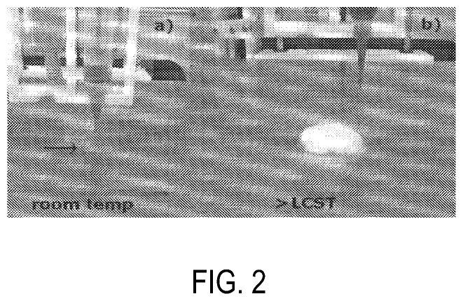

The gel consistency and/or appearance of the hydrogels can change at different temperatures, as demonstrated in FIG. 2. A hydrogel was prepared from 25% VCL, 50% meHA, 5% water, and 15% initiator. FIG. 2, Panel A shows the hydrogel undergoing three-dimensional printing at room temperature. The hydrogel at room temperature is clear. However, at temperatures above the lower critical solution temperature (LCST), the hydrogel appearance can change. The hydrogel material shown in FIG. 2, Panel B is the same material from Panel A but at a temperature above 32° C., which is the LCST for this hydrogel. The hydrogel at temperatures above the LCST are opaque in color due to the cloudy point phase transitions.

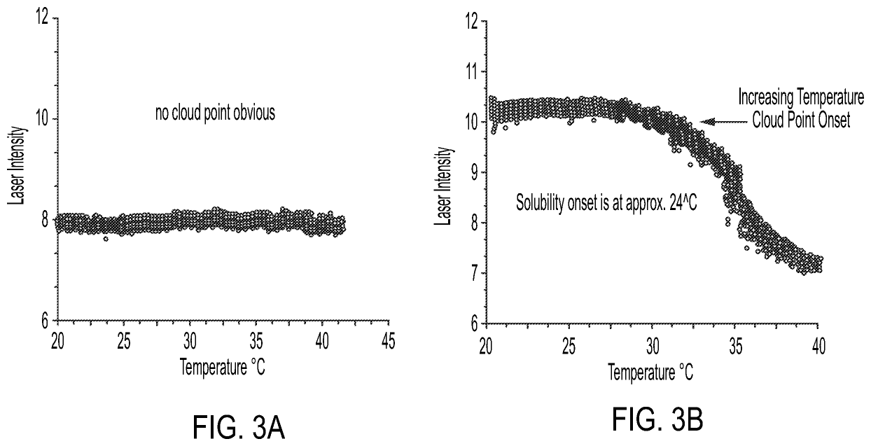

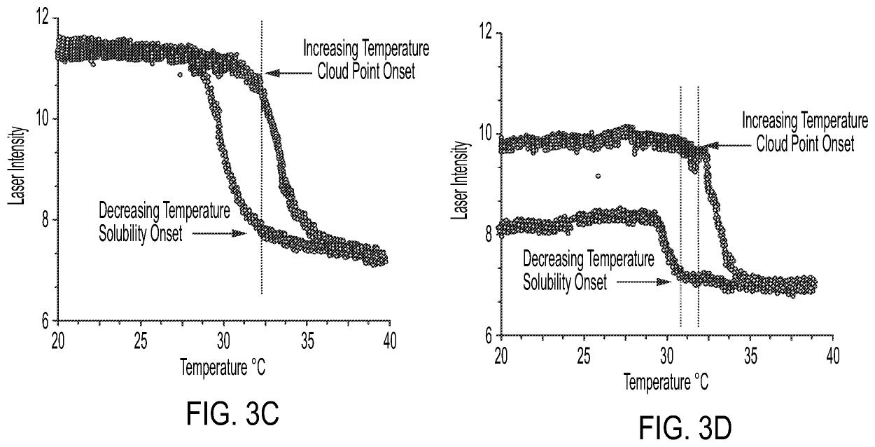

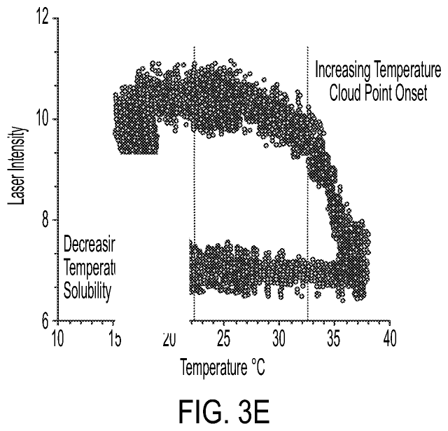

The cloudy point phase transitions were further explored by performing turbidity measurements of meHA and PVCL monomers, along with three hydrogels prepared by polymerizing meHA and PVCL. PVCL-g-HA1 was prepared from 25% VCL, 60% meHA, 10% water, and 5% VA-057 initiator. PVCL-g-HA2 was prepared from 42.5% VCL, 30% meHA, 15% water, and 12.5% VA-057 initiator. PVCL-g-HA3 was prepared from 25% VCL, 50% meHA, 10% wat r, and 15% VA-057 initiator. FIG. 3 contains graphs showing turbidity (i.e., cloudy point) measurements of meHA and PVCL biocompatible composite hydrogels at 2% (w/v) concentration. Panel A shows the turbidity measurement of meHA. Panel B shows the turbidity measurement of PVCL. Panel C shows the turbidity measurement of PVCL-g-HA1. Panel D shows the turbidity measurement of PVCL-g-HA2. Panel E shows the turbidity measurement of PVCL-g-HA3. The monomers (Panels A and B) do not undergo any turbidity phase changes. The composite gels that contain meHA and PVCL (Panels C-E) show the onset of the LCST and the reversibility of the LCST as the temperature is decreased.

### Example 4. Hydrogels with Chondrocytes

### Preparation of Hydrogels

The PVCL-based hydrogels listed in Table 1 were prepared and characterized according to the procedures described above in Example 2.

TABLE 1

| | | Preparation | | | | | |
|---|---|---|---|---|---|---|---|
| | | | | Therapeutic | Cross- | | |
| Sample | Hydrogel Name | PVCL | gmHA | Agent | Linker | Initiator | Solvent |
| 1 | PVCL-g-HA3 | 60% | 25% | — | — | 2.5% | 12.5% |
| 2A | PVCL-c-HA5 | 50% | 35% | — | 1% | 0.5% | 13.5% |
| 2B | PVCL-c-HA6 | 50% | 35% | — | 1% | 0.5% | 13.5% |
| 3 | PVCL-c-HA-ata1 | 40% | 4% | 40% | 1% | 8% | 7% |
| 4 | PVCL-c-HA-ata2 | 40% | 4% | 40% | 1% | 8% | 7% |

In Samples 3 and 4, the therapeutic agent is alpha-tocopherol acrylate (ata). In Samples 2A and 2B, the cross-linker is bisacrylamide. In Samples 3 and 4, the cross-linker is polyethylene oxide diacrylate (PEO-DA). In Samples 1, 2A, B, and 4, the initiator is VA-057 and the solvent is water. In Sample 3, the initiator is AIBN and the solvent is benzene.

The PVCL biomaterial provides a thermo-responsive property that allows the gel to change from a viscous fluid to a gel upon exposure to psychological temperatures. HA is a naturally occurring polysaccharide in the knee joint that provides support, and the therapeutic agent (alpha-tocopherol acrylate (ata)) is a vitamin E derivative that provides an antioxidant property towards diminishing inflammatory cytokine expression under hypoxia.

### Fetal Bovine and Stem (Mesenchymal) Cell Culture:

Articular cartilage samples were harvested from the joint of a fetal bovine calf leg between 5-10 days of age. The samples were washed in phosphate buffer saline (PBS) with 2% antibiotics for 30-60 minutes. Next, the samples were cut into smaller pieces and then digested in 0.1% collagenase for 16-18 hours at 37° C. in an incubator. The chondrocytes were washed afterwards with PBS. The hydrogel samples from Table 1 were sterilized using UV radiation for 16-18 hours. All samples were then mixed in low glucose DMEM supplemented with 10% FBS 25 μg/mL _{L}-ascorbic acid, and antibiotics at 10% (w/v) and 3.65 million cells/milliliter. Low oxygen conditions were employed at 1% oxygen levels using a low oxygen chamber (Heracell 150i, Thermoscientific, USA).

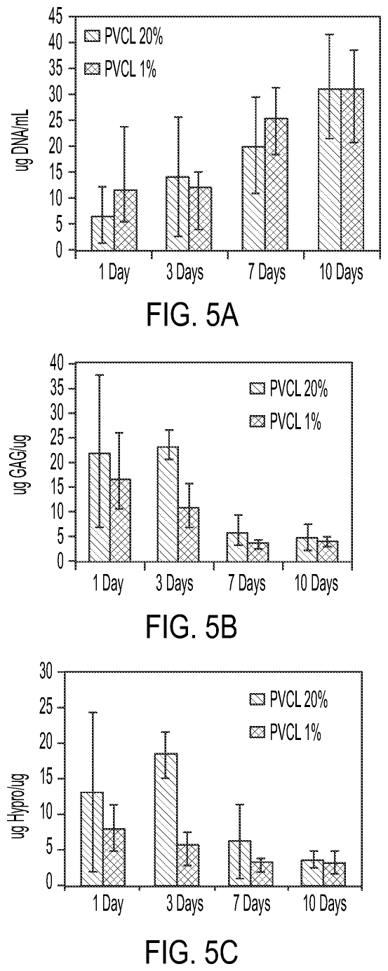

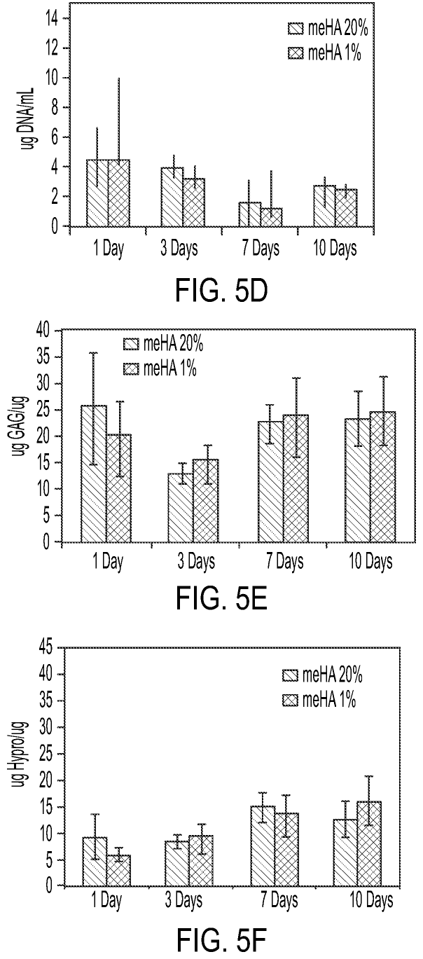

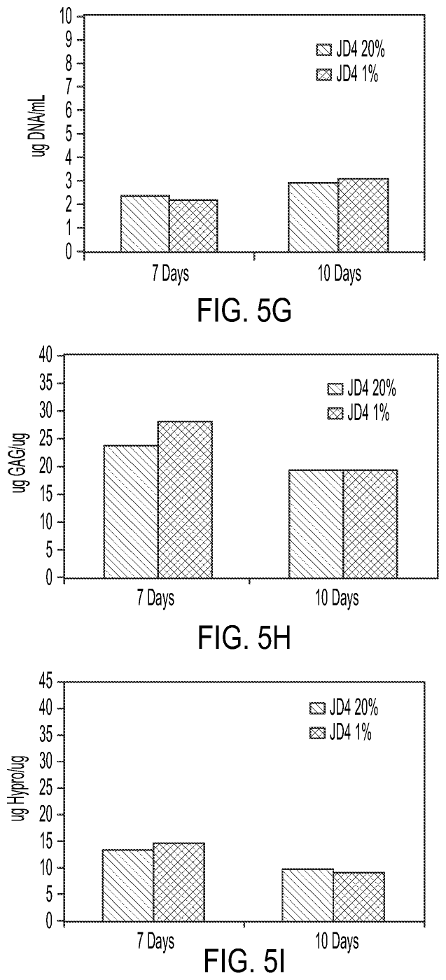

The fetal bovine chondrocyte metabolism profiles for DNA, GAG, and hydroxyproline were determined for cells cultured in monomers and hydrogels as described herein. FIG. 5, left panels (top to bottom), show the DNA, GAG, and hydroxyproline metabolism profiles for cells cultured in PVCL at 20% and 1% O₂ levels. FIG. 5, middle panels (top to bottom), show the DNA, GAG, and hydroxyproline metabolism profiles for cells cultured in meHA at 20% and 1% O₂ levels. FIG. 5, right panels (top to bottom), show the DNA, GAG, and hydroxyproline metabolism profiles for cells cultured in a PVCL-g-HA-aTA (labeled as JD4) at 20% and 1% O₂ levels. The effect of 20% O₂ on PVCL samples indicate higher DNA and hydroxyproline (collagen) values after 10 days of culture. Low oxygen conditions had significant effect on GAG cultured on meHA and the JD4 biocompatible copolymers. Larger hydroxyproline values were present at 20% and 1% O₂ in JD4 gels versus meHA gels, showing a chondroprotective effect on chondrocyte cells under hypoxia (1% O₂). All data sets (n=3) are presented as mean±STD samples. Overall differences in the group are noted over sample bar using: *p<0.05; **p<0.0003; ***p<0.0001.

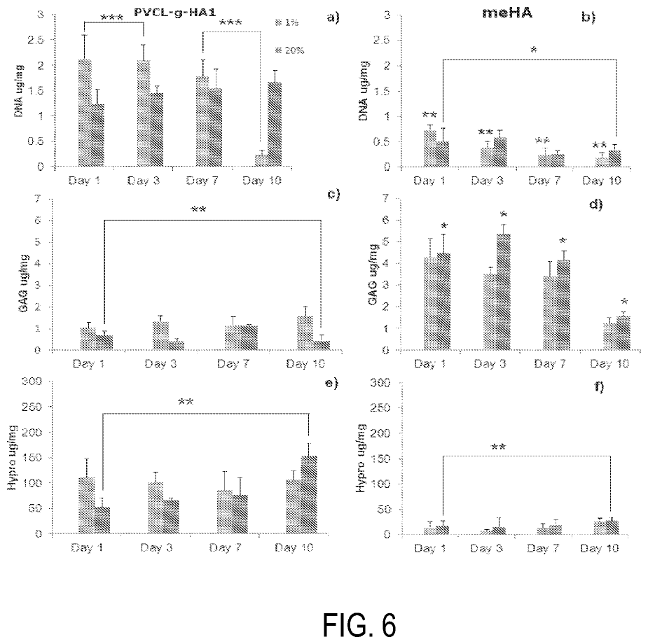

FIG. 6, left panels (top to bottom), show the DNA (panel a), GAG (panel c), and hydroxyproline (panel e) metabolism profiles for cells cultured in PVCL-g-HA1 at 20% and 1% O₂ levels. FIG. 6, right panels (top to bottom), show the DNA (panel b), GAG (panel d), and hydroxyproline (panel f) metabolism profiles for cells cultured in meHA at 20% and 1% O₂ levels. The effect of 20% O₂ on PVCL-g-HA1 samples indicate higher DNA and hydroxyproline values after 10 days of culture. Low oxygen conditions had a significant effect on GAG cultured on meHA. Larger hydroxyproline values were present at 20% and 1% O₂ in PVCL-g-HA1 gels versus meHA gels. All data sets (n=4) are presented as mean±STD samples. Overall differences in the group are noted over sample bar using: *p<0.05; **p<0.0003; ***p<0.0001.

### Human Cartilage Cells (C28 12) Cell Culture:

Static cell culture of hydrogels in 12-well plates cells were encapsulated into hydrogels using a stopcock to inject an average of 2.5×10⁶ cells/mL (DMEM/F12 10% FBS and anti-fungal and antimicrobial). Hydrogel samples were harvested at days 1, 3, 7, and 14, in 20% oxygen (normoxia) and 5% O₂ (hypoxia) incubation. Hypoxia experiments were carried out using a Biospherix® control inside an incubator.

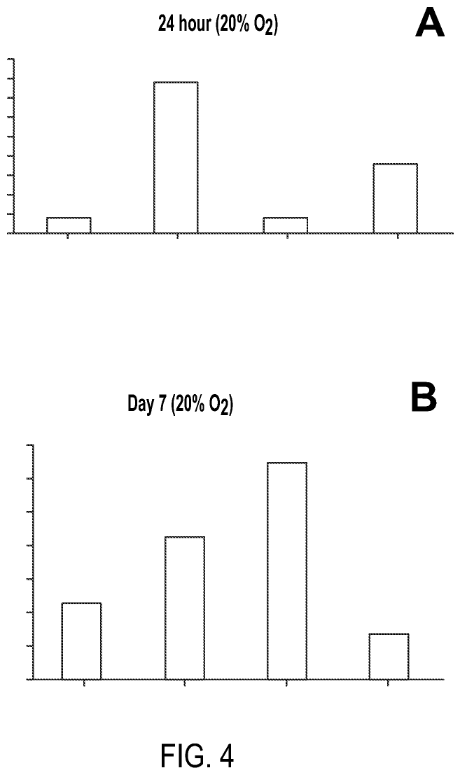

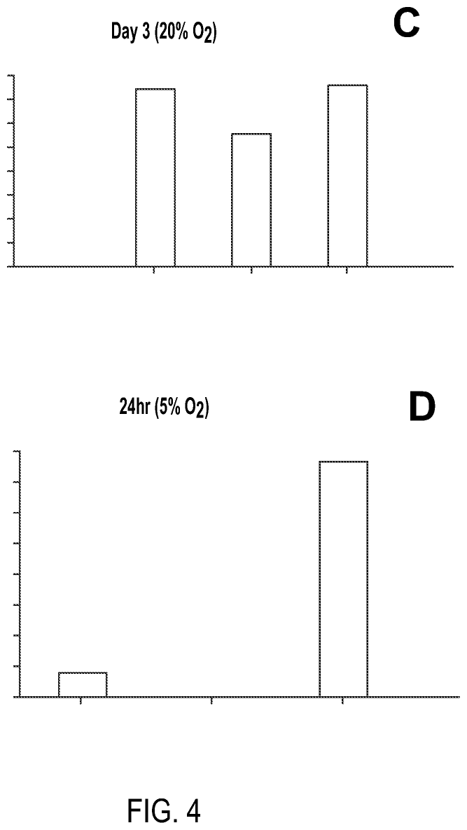

Cell viability measurements of C28/12 human chondrocytes cells were performed under normal conditions (20% O₂) and under hypoxia conditions (5% O₂) (see FIG. 4). An MTT assay was performed to quantify cell proliferation throughout the hydrogel and assess the cell viability of each hydrogel formulation. Under normal conditions, the cells remained viable on most gels at lower cell density. After 24 hours in 5% O₂, gel 4 displayed a high cell density, whereas gel 3 displayed a low cell density.

### Example 5: Evaluation of meHA/PVCL/α-Tocopherol Acrylate

α-tocopherol acrylate (α-TA) was polymerized with two additional macromonomers, PVCL and methacrylic hyaluronic acid. α-TA was prepared by reacting α-tocopherol (α-TP) under inert conditions with acryloyl chloride and tetraethylamine in dichloromethane. The reaction produced a thick yellow liquid which was monitored by TLC and analyzed using NMR and has some hydrophobicity that is challenging when working in aqueous based systems.

Cellular metabolism was evaluated under hypoxia (O₂ 1%) confirming cell viability and modest ECM production inside PVCL-g-HA-α-TA.

### Example 6: Evaluation of Hypoxia on Chondrocyte Metabolism in PVCL/meHA Hydrogels

The effect of chondrocyte growth and proliferation on PVCL/meHA hydrogels at 20% and 1% O₂ levels was investigated to evaluate hypoxia on chondrocyte metabolism on newly prepared thermosensitive injectable PVCL/meHA hydrogels.

### VCL Polymerization

Poly(N-vinylcaprolactam) homopolymers were prepared using the following procedure. VCL was recrystallized in benzene and dried under vacuum. Recrystallized VCL and an initiator were dissolved in benzene or in millipore 18Ω water. When the solvent was benzene the initiator was AIBN, and when the solvent was millipore 18Ω water the initiator was VA-057. The VCL and AIBN or VCL and VA-057 reacted to create PVCL6 and PVCL8 (carboxylic acid end groups), respectively. Table 2 shows synthesis parameters for these reactions. After polymerization the polymers were collected via precipitation in hexane or acetone and dried under vacuum for 48 hours.

Methacrylated Hyaluronic Acid (meHA)

Methacrylated hyaluronic acid was prepared using the following method. Hyaluronan (MW 58 kDA & 1100 kDA) 2% (w/v) was added to methacrylic anhydride in a 20 fold access adjusting to pH 8 using 5N NaOH. The solution was allowed to react overnight at 4° C. and dialyzed against water for 48 hours then lyophilized. Samples of the meHA were rehydrated at 2% or 5% (w/v) for chemical analysis or characterization.

### PVCL/Hyaluronic Acid

VCL was recrystallized in benzene and dried under vacuum. Under an inert environment, recrystallized VCL monomer was allowed to mix with meHA in millipore (80Ω) water at 55° C. After temperature stabilization, VA-057 was added dropwise into the dissolved VCL/meHA mixture and allowed to react overnight. The mixture was precipitated in cold ethanol and dialyzed in DI water for up to 7 days followed by free-drying.

### Hydrogel Synthesis & Characterization

Hydrogels containing PVCL and HA were prepared via free radical polymerization using the water-soluble initiator, VA-057, and polymerizing via the vinyl functional groups in the polymer backbone. All polymerizations were carried out at 55° C., to activate the vinyl groups in the VCL and meHA. The formulations of VCL monomer and meHA (macromonomer) are altered to change the consistency of the gels upon temperature change. Table 2 shows synthesis parameters for PVCL-g-HA and PVCL homopolymers in benzene and water (*denotes PVCL only).

TABLE 2

| | | | | | | | Grafting |
|---|---|---|---|---|---|---|---|
| | VCL | meHA | | | Reaction | Mw* | efficiency |
| Sample | (mol) | (mg) | Initiator | Solvent | time(h) | (g/mol) | (%) |
| PVCL-g-HA1 | 0.2 | 0.3 | VA-057 | Water | 12 | 1.5 × 10⁶ | 54 |
| PVCL-g-HA2 | 0.2 | 6 | VA-057 | Water | 12 | 5.0 × 10⁶ | 49 |
| PVCL-g-HA3 | 0.2 | 0.2 | VA-057 | Water | 12 | 3.0 × 10⁶ | 57 |
| PVCL 6 | 0.2 | — | AlBN | Benzene | 12 | 2.0 × 10⁶ | — |
| PVCL 8 | 0.2 | — | VA-057 | water | 12 | 3.0 × 10⁷ | — |

The free radical polymerization represents a random distribution of monomers along the backbone. Molecular weights and degree of substitution were analyzed using size exclusion chromatography (SEC). ¹H NMR and ATR-FTIR spectroscopy confirmed the formation of PVCL and also the conjugation of meHA. The disappearance of the vinyl proton at 7.5 ppm in ¹H NMR confirmed no residual monomer in PVCL. ¹H NMR VCL, CDCl₃: 7.5-7.4 (1H, m, N—CH vinyl group), 4.3-4.2 (2H, m, CH₂ vinyl group), 3.3 (2H, t, N—CH₂ caprolactam ring), 2.25 (2H, t, COCH₂ caprolactam ring), 1.4-1.2 (6H, m, CH₂ caprolactam ring). ¹H NMR PVCL-3, CDCl₃: 4.5-4 (1H, b, N—CH backbone), 3.5-3.0 (2H, b, N—CH₂ caprolactam ring), 2.5-2.0 (2H, b, COCH₂ caprolactam ring), 2.0-1.0 (8H, b, CH₂ backbone and caprolactam).

ATR-FTIR spectra overlay confirmed the chemical structure of PVCL-g-HA, meHA and HA. A broad absorption at 3350 cm⁻¹ revealed O—H stretching from COOH groups of HA, and characteristic absorption peaks at 1659 cm⁻¹ (C═O stretch) were present. In meHA samples, a vinyl (C═C) is present around 1660 cm⁻¹. For PVCL samples, absorptions around 1631 cm⁻¹ (amide 1), 1480 cm⁻¹ (alpha C—N stretch), and 1350-1500 cm⁻¹ (C—H deformation) were present. NMR spectroscopy was also employed to confirm chemical structure. Notably the NMR spectra for HA controls differ from meHA. The addition of the vinyl group for meHA is confirmed by a doublet peak at 4.4 ppm.

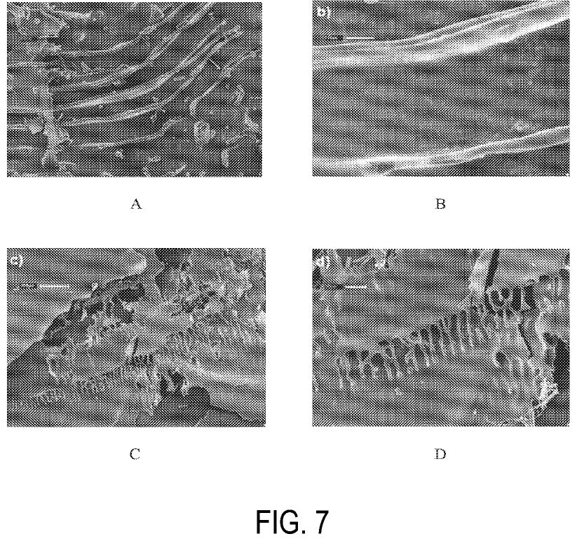

Hydrogel morphology was observed using SEM of lyophilized hydrogels. FIGS. 7A-D are SEM images of (a) PVCL-g-HA1 (scale bar 30 μm), (b) enhanced PVCL-g-HA1 (scale bar 10 μm), (c) PVCL-g-HA3 (scale bar 100 μm), and (d) PVCL-g-HA3 (scale bar 30 μm). The SEM shows structures for PVCL-g-HA1 having distinct changes in phase separation between PVCL and HA with edges. Similar architectures were also seen in PVCL-g-HA2 hydrogels. Contrary to PVCL-g-HA1, PVCL-g-HA3 has sheet-like structures indicating the separation of polymers.

### Turbidity

Turbidity experiments using laser intensity determined the LCST, T_{G}, and reversible solubility temperatures (T_{S}) of PVCL, meHA, and PVCL-g-HA samples. Turbidity-temperature profiles illustrate the LCST of meHA, PVCL-g-HA, and PVCL samples. The correlations between polymer and hydrogel samples are noted by the inflection point on a curve. Samples of composite hydrogels of PVCL-g-HA indicated a consistent LCST between 33-37° C. Typical temperature-turbidity curves of thermosensitive hydrogels depict one way transitions with limited info reported on reversibility temperature parameters. However, the reversibility temperatures present a unique parameter notable to polymer chain interactions and PVCL molecular weight. PVCL-g-HA hydrogels show typical LCST behavior where upon heating a transition from a homogeneous one-phase system becomes a two-phase system comprised of the copolymer suspended within a solvent medium upon increasing temperature.

Experimental values for LCST and gelation temperatures for PVCL-g-HA hydrogels as determined by turbidity and rheological measurements were determined. Table 3 shows lower critical solution temperature (LCST), solubility temperature, and gelation temperature parameters of hydrogel and polymer samples using turbidity apparatus.

TABLE 3

| | LCST | Solubility | Gelation | Swelling |
|---|---|---|---|---|
| Sample Name | (° C.) | Temp (° C.) | Temp(° C.) | Ratio |
| PVCL-g-HA1 | 33 | 33 | 45 | 85 |
| PVCL-g-HA2 | 33 | 31 | 41 | 65 |
| PVCL-g-HA3 | 34 | 24 | 45 | 70 |
| PVCL 6 | 37 | 34 | 42 | — |
| PVCL 8 | 37 | 34 | 43 | — |

A cloudy point was observed at 33-34° C. upon heating within the experimental temperature range for each PVCL-g-HA solution at a concentration of 0.5% (w/v). The PVCL homopolymer controls, PVCL6 and PVCL8, were also observed to undergo a cloud point transition at 37° C. Conversely, the meHA homopolymer control did not exhibit a cloud point transition which indicates that the LCST behavior observed for the PVCL-g-HA copolymers results from the grafted PVCL polymer chains. The meHA sample displayed an even turbidity having laser intensity of 8. The observed turbidity is reversible upon cooling. Upon cooling, PVCL-g-HA1, PVCL-g-HA2, and PVCL-g-HA3 solutions transition from heterogeneous suspensions to homogeneous solutions at 33° C., 31° C., and 22° C. respectively.

### Temperature-Controlled Rheology

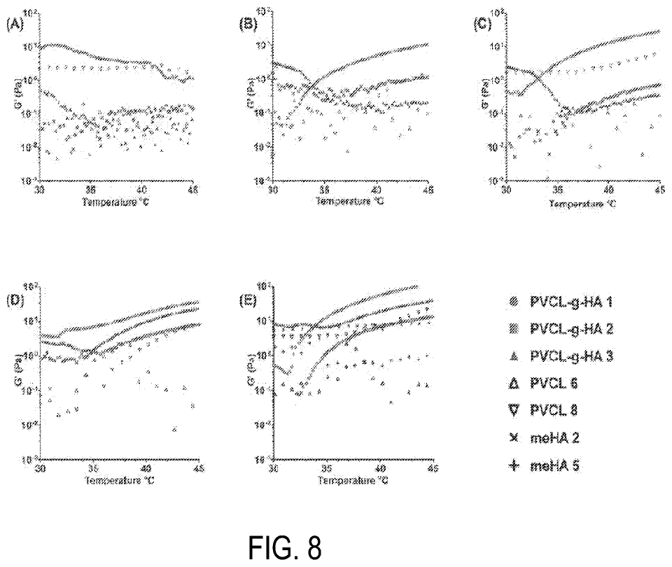

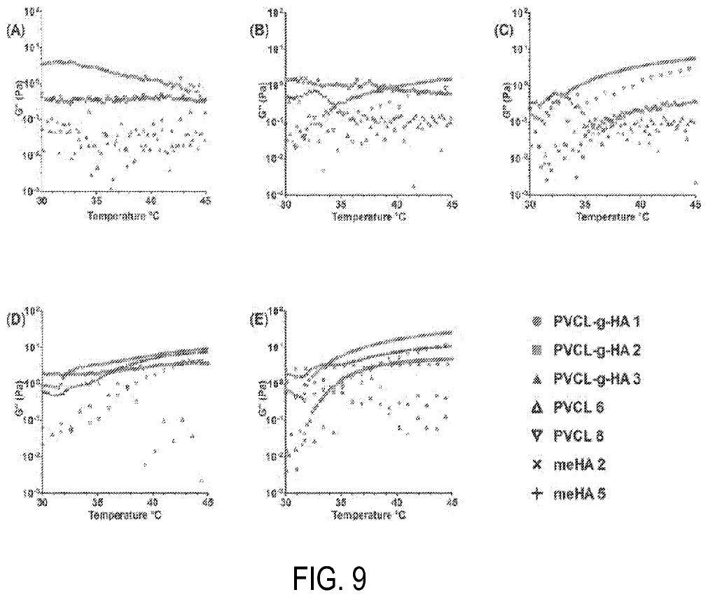

Elastic (G′) and viscoelastic (G″) modulus measured using parallel plate geometry was used to create temperature versus stress-strain profiles for PVCL-g-HA, meHA, and PVCL homopolymers. FIGS. 8 A-E are graphs showing elastic modulus (G′) versus temperature from temperature ramp rheology experiments at constant strain of 1.0% and angular frequency of 1.0 rad/s for PVCL-g-HA1, PVCL-g-HA2, PVCL-g-HA3, PVCL6, PVCL8, and meHA samples at (A) 1% (w/v), (B) 2% (w/v), (C) 3% (w/v), (D) 4% (w/v), and (E) 5% (w/v). FIGS. 9 A-D are graphs showing viscous modulus (G″) versus temperature from temperature ramp rheology experiments at constant strain of 1.0% and angular frequency of 1.0 rad/s for PVCL-g-HA1, PVCL-g-HA2, PVCL-$-HA3, PVCL6, PVCL8, and meHA samples at (A) 1% (w/v), (B) 2% (w/v), (C) 3% (w/v), (1l) 4% (w/v), and (E) 5% (w/v).

Table 4 shows the correlation between sample concentration and elastic and viscoelastic moduli at the LCST.

TABLE 4

| | | G′ (Pa) | | | | | G″ (Pa) | | | | |
|---|---|---|---|---|---|---|---|---|---|---|---|
| Sample | LCST (° C.) | 1% | 2% | 3% | 4% | 5% | 1% | 2% | 3% | 4% | 5% |
| PVCL-g-HA1 | 33 | 7.7 | 0.5 | 1.4 | 6.1 | 5.5 | 3.3 | 0.1 | 0.7 | 2.8 | 3.0 |
| PVCL-g-HA2 | 33 | 0.02 | 0.6 | 0.05 | 0.8 | 0.3 | 0.4 | 1.0 | 0.02 | 1.8 | 0.5 |
| PVCL-g-HA3 | 34 | 0.06 | 0.5 | 0.4 | 1.5 | 6.6 | 0.0 | 0.3 | 0.2 | 1.1 | 3.5 |
| PVCL 6 | 37 | 0.5 | 1.3 | 1.5 | 2.4 | 1.0 | 0.2 | 0.6 | 0.7 | 0.9 | 0.9 |
| PVCL 8 | 37 | 2.4 | 0.4 | 2.4 | 0.6 | 2.8 | 0.5 | 0.6 | 0.7 | 0.7 | 2.6 |

Table 5 shows the correlation between sample concentration and elastic and viscoelastic moduli at the gelation temperature.

TABLE 5

| | Gelation | G′ (Pa) | | | | | G″ (Pa) | | | | |
|---|---|---|---|---|---|---|---|---|---|---|---|
| Sample | Temp (° C.) | 1% | 2% | 3% | 4% | 5% | 1% | 2% | 3% | 4% | 5% |
| PVCL-g-HA1 | 45 | 1.3 | 11 | 29 | 37 | 123 | 0.3 | 1.6 | 5.6 | 9.2 | 27 |
| PVCL-g-HA2 | 41 | 0.1 | 0.8 | 0.4 | 4.9 | 8.6 | 0.4 | 0.7 | 0.2 | 3.4 | 4.2 |
| PVCL-g-HA3 | 45 | 0.06 | 0.2 | 0.4 | 24 | 40 | 7⁻³ | 0.1 | 0.1 | 8.0 | 11 |
| PVCL 6 | 42 | 0.07 | 0.5 | 0.04 | 0.2 | 0.06 | 0.1 | 0.1 | 0.01 | 0.4 | 0.01 |
| PVCL 8 | 43 | 1.4 | 1.5 | 6.5 | 8.8 | 25 | 0.9 | 1.2 | 2.4 | 4.6 | 12 |

At LCST, as concentration amounts increased for PVCL-g-HA2, G′ and G″ values remained below 1.0 Pa. Similar trends were observed with PVCL-g-HA2 samples at 1-3% (w/v). However, at concentrations of 5% (w/v), PVCL-g-HA1 demonstrated higher G′ and G″ at the LCST and Ta. A notable higher linear increase of G′ with temperature is observed in PVCL-g-HA1 at all concentrations. Inconsistent moduli were noted with PVCL-g-HA2, the G′ demonstrated a non-continuum rate as temperature change showing non-uniformity. In comparison, meHA hydrogel modulus was consistent with polymer chain association upon shearing the sample. Homopolymer samples displayed increased concentration of PVCL6 polymers reported the highest G′ and G″ at 4% (w/v) at 2.4 Pa and 0.9 Pa, respectively. Interestingly, PVCL8 had the high G′ and G″ at 5% (w/v) at 2.8 and 2.6 Pa at the LCST of 34° C., respectively. Opposite trends were noticed in PVCL-g-HA1 at LCST 33° C., showing moduli at lower concentrations (1% w/v) versus higher concentration. The highest G′ and G″ values for PVCL-g-HA1 were 7.7 Pa and 3.3 Pa. Moduli measured at T_{G} had higher trends with increasing concentrations, except for PVCL6. A designated T_{G} was observed for PVCL6 at 42° C., however the homopolymer did not reach moduli values higher than 0.5 Pa (G′) and 0.4 (G″) at 2%/(w/v) and 4% (w/v). Linear moduli increases were observed for PVCL8 at 43° C., notably increases with concentration ranged from 1.4 Pa to 25 Pa for G′. Additionally for PVCL8, G″ moduli between 1% (w/v)-5% (w/v) ranged from 0.9-12 Pa at T_{G}. Moduli near the T_{G} for PVCL-g-HA2 and PVCL-g-HA3 were concentration dependent being the highest at 5% (w/v); Observations at 3% (w/v) demonstrated a decrease in moduli of about 50% occur for PVCL-g-HA2 and PVCL-g-HA3, then an increase at 4% (w/v) and 5% (w/v).

### Cell Viability

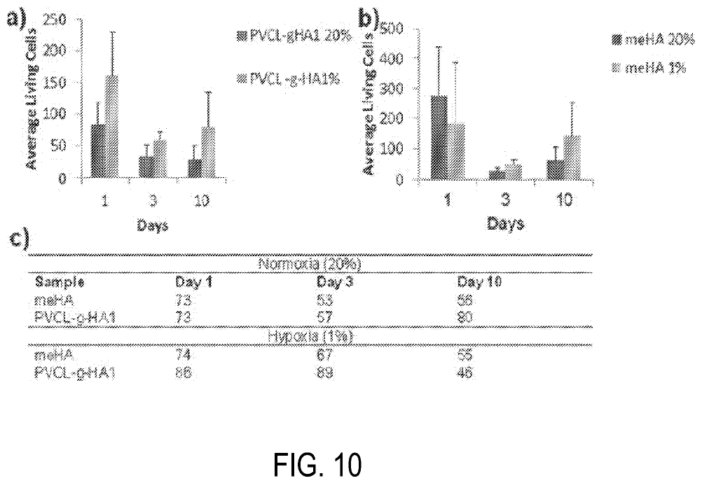

Extracellular matrix production is an integral part of developing 3D constructs to support cell growth and regeneration. Chondrocytes were harvested after 1, 3, 7, and 10 days at 20% and 1% O₂ and cell viability, DNA, GAG, and collagen deposition on hydrogel samples were assessed. Cell viability was observed using confocal microscopy after 1, 3, and 10 days for samples meHA and PVCL-g-HA. Viability of cells was maintained for all samples throughout the 10 day time period. However, cell viability at 1% O₂ levels remained higher than that of 20% O₂ levels in PVCL-g-HA for each time point. FIGS. 10 A-C show results of cell viability analysis of average living cells using calcein AM and EthD-1 of (A) PVCL-g-HA1 at 20% and 1%, (B) meHA at 20% and 1%, and (C) percentage of cell viability at normoxia and hypoxia from fluorescent images of green cells/red cells. Values are recorded in mean±SD (n 5 4). PVCL-g-HA hydrogels reached a maximum of 89% on the third day of observance. Higher cell viability was also noted on meHA samples at 1% O₂ levels than at 20% O₂ levels, with the peak value at 74% on the first day of observance (FIG. 10C).

Hypoxia (1% O₂) had the greatest effect on chondrocytes cultured in PVCL-g-HA hydrogels at 1 and 3 days. The use of 1% O₂ was chosen to mimic the diseased state affiliated with the onset of diseased or damaged articular cartilage. ECM production by chondrocytes was highest in meHA samples at 7 and 10 days, with little difference in oxygen tension. Hypoxia had a smaller effect on cell metabolism than expected. Chondrocyte metabolism cultured in alginate beads suppresses ECM production of collagen, and metabolic synthesis is contingent upon pO₂. This supports the oxygen level concentration in cartilage zones and the correlation between RNA and DNA synthesis. The effect of hypoxia on cell culture improves GAG and hydroxyproline. In native cartilage, chondrocytes in the avascular tissue are highly affected by hypoxia which results in a loss of call viability and mechanical integrity. Using 3D matrices to culture chondrocytes in vitro have shown to provide a viable support for fetal bovine chondrocyte metabolism and ECM synthesis.

### Biochemical Analysis

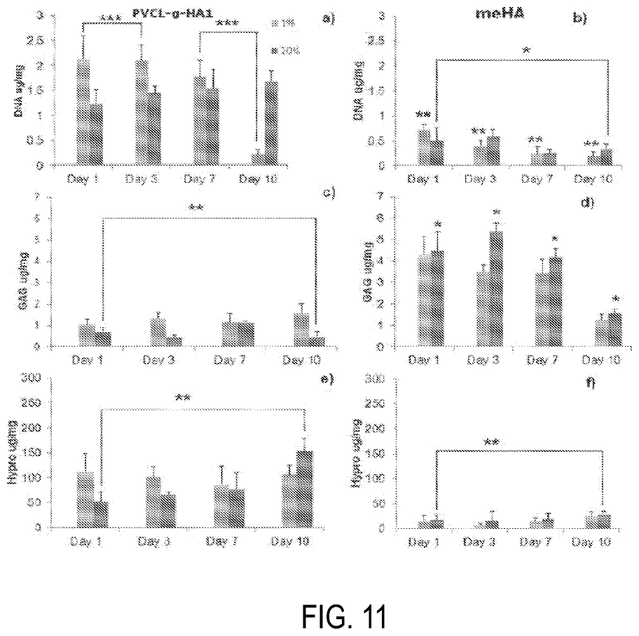

ECM synthesis was evaluated under normal and hypoxic environments for up to 10 days of cell culture. FIGS. 11 A-E show DNA and chondrocyte ECM synthesis on PVCL-g-HA and meHA hydrogels at 20% and 1% oxygen levels. Samples were analyzed on days 1, 3, 7, and 10. All data sets are presented as mean±SD for n=4 samples. Observations of DNA content were significantly higher in PVCL-g-HA1 (1.7±μg/mg sample) than that of meHA (0.2±sg/mg) at 20% O₂ levels. However at 1% O₂, PVCL-g-HA1 chondrogenic DNA synthesis averaged 1.5 mg/mg, with a decrease on day 10 of 0.2 mg/mg. DNA production trends in meHA samples remained constant under normal and hypoxic conditions. At normal oxygen conditions of meHA chondrogenic cell culture samples averaged around 0.1±0.055 mg/mg. The highest DNA content (1.0±1.7 mg/mg) for meHA was on Day 10 in 1% O₂. The evaluation of GAG content remained constant throughout PVCL-g-HA1 gels at 1% O₂ environments with the highest amount of 1.6±0.5 sg/mg on day 10. GAG production was highest on PVCL-g-HA1 on day 7 at 20% O₂ (1.1±0.1 sg/mg). However in 20/a O₂, GAG contents on meHA were highest on day 3 at 5.4±0.4 μg/mg and lowest on day 10 under hypoxia (1.2±0.2 μg/mg). The hydroxyproline assay was performed to analyze collagen synthesis on PVCL-g-HA and meHA samples. A notable increase was present in collagen dry weight amounts on both hydrogel samples. The composite hydrogels of PVCL-g-HA confirmed linear increases amounts of collagen with the highest values (153±25 μg/mg) on day 10. Conversely, hydroxyproline values at day 10 in meHA were 28±5 sg/mg when cultured at normal oxygen levels. Collagen synthesis under hypoxic (1% O₂) conditions were also higher in PVCL-g-HA1 versus meHA. PVCL-g-HA hydroxyproline amounts were highest on day 1 at 111±37 mg/mg, after day 10 collagen deposition was noted as 106±18 mg/mg. Remarkably, meHA collagen values were highest on day 10 remaining constant at 28±5 mg/mg (20%) and 26±5 mg/mg (1%). These results depicted a 10-fold collagen synthesis increase on temperature-sensitive PVCL-g-HA1 hydrogels and meHA hydrogel.

Random chain dispersion of free radicals, grafting percentage, and the solvent used in polymerization affect grafting DPI rates. Water solvents using VA-057 produce higher MW and broader DPI to the polarity and larger chain entanglements. Swelling and grafting ratios also contribute to chain entanglement that may affect molecular structure interpretation.

Thermodynamically, LCST behavior arises when sufficient energy, typically in the form of heat energy, is input into the system driving phase separation of the copolymer from the solvent. With increasing temperature the polymer gains affinity for itself over the solubilizing medium. This lower critical phenomenon has also been observed in polymer melts in the form of a lower disorder-to-order transition (LDOT). Because the PVCL homopolymer controls also exhibit LCST behavior in aqueous solution, the thermodynamically driven LCST behavior in the PVCL-g-HA copolymer solutions can be attributed to dehydration of the PVCL grafted chains resulting in fast agglomeration of the copolymers into an insoluble phase. There is no observable correlation between the LCST behavior and the chemical composition of the PVCL-g-HA copolymers.

Mechanical properties of hydrogels can be affected by several parameters such as polymer molecular weight, sample concentration, chain entanglement, and cross-linker amounts. However, an intricate balance of polymer molecular weight and concentration must coexist to support tissue engineering strategies. The elastic modulus and viscoelastic modulus was studied to further understand the physicochemical properties of PVCL-g-HA gels. meHA displayed an increase in the elastic modulus, G′ (about 2.0 Pa) and the viscous modulus, G″ (about 1.5 Pa) between 22-34° C. PVCL6 showed a dramatic decrease in temperature dependence of G′ and G″ at about 38° C. that is indicative of insolubility (LCST) in addition to formation of gelation occurring. PVCL 6 has high viscosity but still remained in solution at 5% (w/v). Shearing rates of PVCL8 exhibited a steady increase in both G′ and G″ after about 35° C. This was consistent with polymer chain association, the PVCL8 chains interact leading to increases in the elasticity and viscosity of the gel. The initial decrease in G′ and G″ indicated polymer chain dissociation of PVCL-g-HA1. However, at about 32° C. the polymer chains began to interact resulting in increasing G′ and G″. PVCL-g-HA2 initially behaved as a solution at about 32° C.; there is a sudden increase in G′ and G″. The crossover point at 36° C. may indicate a solution-to-gel transition. The consistent G′ and G″ values of PVCL-g-HA2 at 3% (w/v) suggested limited molecular overlap that maybe affiliated with lower molecular weight of PVCL. The advantage of using temperature-controlled rheology to study stress/strain relationships is to further understand the tunable of PVCL-g-HA gels upon temperature change.

Cell laden hydrogels with optimum mechanical properties comparable to native cartilage are ideal for developing cartilage scaffolds and tissue engineered articular cartilage constructs. Although the modulus of the PVCL-g-HA gels in the current study were below that of native cartilage, materials used for filling focal articular cartilage defects will bear load in tandem with surrounding tissues. Thus, such materials are useful for articular cartilage repair.

The hydrogels disclosed herein can be used as injectable hydrogels due to their enhanced elastic and viscoelastic moduli upon To and concentration. Furthermore, the PVCL and HA segments are both biocompatible as noted by the increase in DNA content.

The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions, methods, and aspects of these compositions and methods are specifically described, other compositions and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A biocompatible copolymer, comprising:
a first plurality of monomers each comprising a polysaccharide or derivative thereof;
a second plurality of monomers each comprising a therapeutic agent or derivative thereof;
a third plurality of monomers each comprising a thermo-responsive monomer; and
an acrylamide-containing cross-linking agent.

2. The biocompatible copolymer of claim 1, wherein each of the monomers and the crosslinking agent comprise a vinyl functional group, and wherein the vinyl functional groups form the backbone of the biocompatible co-polymer.

3. The biocompatible copolymer of claim 1, wherein the polysaccharide or derivative thereof comprises hyaluronic acid, methacrylated hyaluronic acid, or glycidyl methacrylated hyaluronic acid.

4. The biocompatible copolymer of claim 1, wherein the thermo-responsive monomer comprises n-vinylcaprolactam.

5. The biocompatible copolymer of claim 1, wherein the therapeutic agent or derivative thereof comprises an anti-osteoarthritic agent or derivative thereof.

6. (canceled)

7. The biocompatible copolymer of claim 1, wherein the acrylamide-containing crosslinking agent is selected from the group consisting of bisacrylamide and poly(ethyleneoxide diacrylamide).

8. The biocompatible copolymer of claim 1, further comprising a surface adhesion protein.

9. (canceled)

10. A hydrogel implant material, comprising the biocompatible copolymer of claim 1 .

11. The hydrogel implant material of claim 10, wherein the composition is a liquid at temperatures below about 32° C. or wherein the composition is a gel at temperatures from above about 32° C.

12. (canceled)

13. The hydrogel implant material of claim 10, further comprising an additional therapeutic agent, wherein the additional therapeutic agent is an anti-inflammatory agent or an anti-infective agent.

14. (canceled)

15. The hydrogel implant material of claim 10, further comprising a plurality of chondrocytes.

16. The hydrogel implant material of claim 10, wherein the implant material comprises the form of a joint meniscus or fragment thereof.

17. A method of treating or preventing joint damage or osteoarthritis in a subject, comprising:
administering to a joint in the subject an effective amount of the hydrogel implant material of claim 10 .

18. The method of claim 17, wherein the joint is a knee joint.

19. The method of claim 17, wherein the composition is administered by intra-articular injection.

20. The method of claim 17, wherein the hydrogel implant material is in the form of a joint meniscus or fragment thereof and wherein the administering step is surgical implantation.

21. The method of claim 20, wherein the meniscus or fragment thereof is produced using three dimensional printing.

22. A co-polymerizable composition, comprising:
n-vinyl caprolactam;
methacrylated hyaluronic acid;
a therapeutic agent or derivative thereof containing a vinyl functional group; and
an acrylamide containing crosslinking agent.

23. The composition of claim 22, further comprising a polymerization initiator.

24.-30. (canceled)

31. A method for making a hydrogel implant, comprising:
placing the co-polymerizable composition of claim 22 within a three-dimensional printing device; and
polymerizing the co-polymerizable composition in one or more printing cycles to produce a hydrogel implant.
